(19) United States
(12) Patent Application Publication
(10) Pub. No.: US 2014/0171856 A1
(43) Pub. Date: Jun. 19, 2014

(54) Applications for Physical-Layer Security

(71) Applicants: Georgia Tech Research Corporation, Atlanta, GA (US); Whisper Communications, LLC, Atlanta, GA (US)

(72) Inventors: Steven W. McLaughlin, Decatur, GA (US); Willie K. Harrison, Colorado Springs, CO (US); Jeffrey McConnell, Marietta, GA (US); Cenk Argon, Chapel Hill, NC (US)

(73) Assignees: Georgia Tech Research Corporation, Atlanta, GA (US); Whisper Communications, LLC, Atlanta, GA (US)

(21) Appl. No.: 13/962,777

(22) Filed: Aug. 8, 2013

Related U.S. Application Data
(60) Provisional application No. 61/680,868, filed on Aug. 8, 2012.
(60) Provisional application No. 61/680,871, filed on Aug. 8, 2012.
(60) Provisional application No. 61/680,874, filed on Aug. 8, 2012.

(51) Int. Cl. A61M 5/172; G08C 17/02; G06Q 30/02; H04W 12/02

(52) U.S. Cl. CPC A61M 5/1723; H04W 12/02; G08C 17/02; G06Q 30/0261
USPC 604/66; 726/26; 340/870.01; 705/14.58

(57) ABSTRACT

Applications for physical layer security are disclosed. One such application is a system comprising a medical sensor device and a wireless communication module. The medical sensor device is operable to generate data representative of a condition of a patient. The wireless communication module is operable to transmit, on a wireless communication channel, the generated data representative of the condition of the patient. The system also includes a physical layer security module residing at a physical layer of the wireless communication module. The physical layer security module is operable to provide a secrecy zone around the physical layer security module by transforming the generated data such that transmission of the generated data is secured from interception by an eavesdropper on the wireless communication channel.

### CROSS REFERENCE TO RELATED APPLICATIONS

This application claims the benefit of U.S. Provisional Application No. 61/680,874, filed Aug. 8, 2012, and of U.S. Provisional Application No. 61/680,868, filed Aug. 8, 2012, and of U.S. Provisional Application No. 61/680,671, filed Aug. 8, 2012, each of which is hereby incorporated by reference herein.

### FIELD OF THE DISCLOSURE

The present disclosure relates to data communication, and more specifically, to secure communication at the physical layer.

### BACKGROUND

Conventional methods of providing secure communication over a channel use cryptography. Cryptography relies on the existence of codes that are “hard to break”: that is, one-way functions that are believed to be computationally infeasible to invert. Cryptography has become increasingly more vulnerable to an increase in computing power and to the development of more efficient attacks. Furthermore, the assumptions about the hardness of certain one-way functions have not been proven mathematically, so cryptography is vulnerable if these assumptions are incorrect.

Another weakness of cryptography is the lack of precise metrics or absolute comparisons between various cryptographic algorithms, to show the tradeoff between reliability and security as a function of the block length of plaintext and ciphertext messages. Instead, a particular cryptographic algorithm is considered “secure” if it survives a defined set of attacks, or “insecure” if it does not.

Cryptography as applied to some media (e.g., wireless networks) also requires a trusted third party as well as complex protocols and system architectures. Therefore, a need exists for these and other problems to be addressed.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure.

FIG. 1 is a block diagram of a communication system that provides physical layer security, according to some embodiments described herein.

FIG. 2 is a system diagram in which a medical device uses the secure physical layer of FIG. 1, according to some embodiments described herein.

FIG. 3 is another system diagram in which a medical device uses the secure physical layer of FIG. 1, according to some embodiments described herein.

FIG. 4 is yet another system diagram in which a mobile medical device uses the secure physical layer of FIG. 1, according to some embodiments described herein.

FIG. 5 is another system diagram in which a mobile medical device uses the secure physical layer of FIG. 1, according to some embodiments described herein.

FIG. 6 is a system diagram of an electronic access system having the secure physical layer of FIG. 1, according to some embodiments described herein.

FIG. 7 is a system diagram of another electronic access system having the secure physical layer of FIG. 1, according to some embodiments described herein.

FIGS. 8A and 8B are system diagrams of additional electronic access systems having the secure physical layer of FIG. 1, according to some embodiments described herein.

FIG. 9 is a system diagram of yet another electronic access system having the secure physical layer of FIG. 1, according to some embodiments described herein.

FIG. 10 is a system diagram of a location-based marketing system having the secure physical layer of FIG. 1, according to some embodiments described herein.

FIG. 11 is a messaging diagram showing operation of a location-based marketing system having the secure physical layer of FIG. 1, according to some embodiments described herein.

FIG. 12 is a hardware block diagram of an embodiment of a secure communication device having the secure physical layer of FIG. 1, according to some embodiments described herein.

### DETAILED DESCRIPTION

Disclosed herein are inventive applications for a secure physical layer for communication between devices. One such application involves secure wireless communications between a medical device and other devices such as a mobile device, a monitoring station, or a reporting station. Another such application involves secure wireless communications between components of electronic access systems, such as transmission of credentials from a mobile device to a credential reader, which in turn communicates with an access mechanism such as a lock. Yet another such application involves location-based marketing, where physical-layer security protects communication of credentials and digital offers/coupons between a mobile device and various other devices such as beacons, tags, and a location-based marketing server.

In the applications described herein, data is secured against eavesdropping at the physical layer of a communication system. A transmitter provides security at the physical layer (referred to herein as “physical-layer security”) by transforming user data in a manner that produces a bit error rate of about one-half at an eavesdropper receiving the secure bit stream. The transform used by a secure physical layer exploits characteristics of the communication channel in a manner that prevents unintended receivers (referred to herein as “eavesdroppers”) from obtaining partial or complete information about the transmitted user data. Security is guaranteed because a one-half bit error rate means a bit decoded by the eavesdropper is as likely to be incorrect as correct. A “friendly” or “intended” receiver recovers the transmitted user data by reversing the specific transformation process used in the transmitter. Notably, some embodiments of the secure physical layer disclosed herein are keyless, where conventional security mechanisms at a higher layer typically use keys.

The secure physical layer embodiments of described herein can be used with secure error correction codes, which are known to a person of ordinary skill in the art to provide physical layer security. One non-limiting example of a secure error correction code is a punctured error correction code. Another non-limiting example of a secure error correction code is a low density parity check (LDPC) code. One class of LPDC codes is disclosed in “Secure Communication Using Error Correction Codes”, U.S. 20100275093, which is hereby incorporated herein by reference. Another non-limiting example of a secure error correction code is a non-systematic error correction code. One class of non-systematic error correcting codes is disclosed in “Secure Communication Using Non-Systematic Error Control Codes”, U.S. 20110246854, which is hereby incorporated herein by reference.

The secure physical layer embodiments of described herein can be also be used with any physical layer pre-processing that provides physical layer security. One example of a physical layer security pre-processor is an arrangement of rate-1 non-recursive convolutional encoders in series with permuters as disclosed in “Pre-Processor for Physical Layer Security”, U.S. Ser. No. 13/908,000, which is hereby incorporated herein by reference.

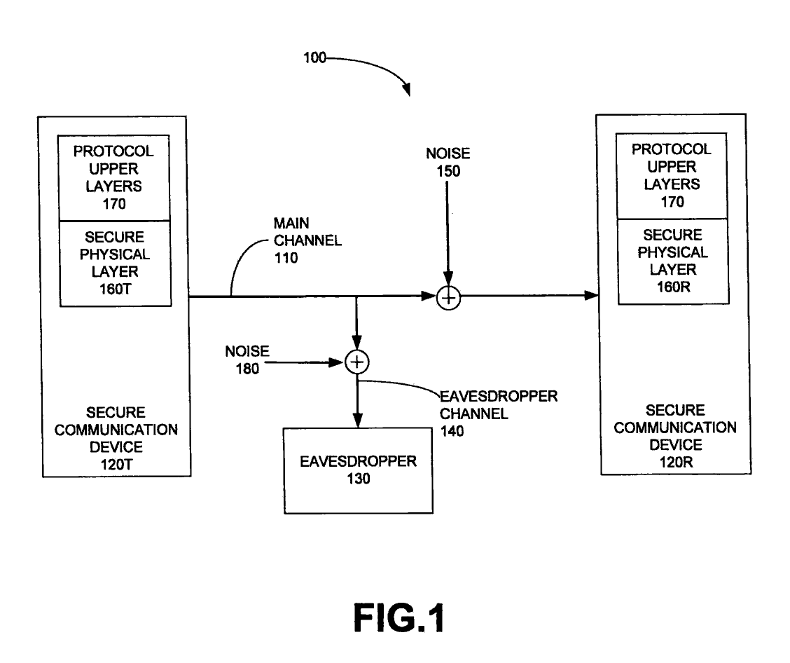

FIG. 1 is a system diagram of a transmitter device and a receiver device cooperating to provide physical layer security. Communication system 100 includes two parties that communicate over a main channel 110: secure communication device 120T, operating as a transmitter; and secure communication device 120R, operating as a receiver. Although transmit and receive operations are discussed separately herein, a person of ordinary skill in the art would understand that some embodiments of device 120 have both transmitter and receiver functionality.

System 100 accounts for another device 130 (an “eavesdropper”) which may listen to (eavesdrop on) transmissions on main channel 110, over an eavesdropper channel 140. Eavesdropper 130 is passive with respect to main channel 110, i.e., eavesdropper 130 does not jam main channel 110, insert bits on main channel 110, etc. In some embodiments, main channel 110 and eavesdropper channel 140 are wireless. In one of these embodiments, secure transmitter 120T and secure receiver 120R are implemented using radio frequency identification (RFID) tags. In other embodiments, main channel 110 and eavesdropper channel 140 are wired (wireline) channels.

Main channel 110 is subject to a noise input 150. As a result, communication from secure transmitter 120T to secure receiver 120R over main channel 110 is not error-free. The performance of main channel 110 can be described in terms of a bit error rate (BER) at secure receiver 120R, which can also be understood as a probability of error (p_{M}) at receiver 120R. Considering a single bit, the probability of secure receiver 120R seeing a 1 when secure transmitter 120T actually sent a 0, or seeing a 0 when transmitter 120T actually sent a 1, is p_{MAIN}. Conversely, the probability of secure receiver 120R seeing a 1 when secure transmitter 120T actually sent a 1, or seeing a 0 when transmitter 120T actually sent a 0, is 1−p_{MAIN}.

A secure physical layer 160T residing in secure transmitter 120T conveys information across main channel 110, where it is recovered by a secure physical layer 160R residing in secure receiver 120R. Though not discussed in detail herein, secure communication device 120 may implement other layers above secure physical layer 160, for example a Media Access Control (MAC) layer, a network layer, a transport layer, a session layer, etc. Such layers are depicted in FIG. 1 as protocol upper layers 170.

As a physical layer, secure physical layer 160 uses techniques known to a person of skill in the art, such as bit mapping, modulation, line coding, etc., to process data into a format that is suitable for the physical characteristics of main channel 110, and to transmit the processed data on main channel 110. Secure physical layer 160 may also use techniques such as channel coding and/or error correction to convey information in a manner which takes into account noise input 150, thus reducing p_{MAIN} as compared to performance without such techniques.

As noted earlier, eavesdropper 130 uses eavesdropper channel 140 to intercept communications between secure transmitter 120T and secure receiver 120R. Eavesdropper 130 then decodes intercepted data in an attempt to recover user data conveyed from secure transmitter 120T and secure receiver 120R. However, eavesdropper channel 140 is subject to a noise input 180 with characteristics different from noise input 150. The probability of error at eavesdropper 130 is referred to herein as p_{EVE}. Security is achieved whenever p_{EVE} is about one-half, since in this scenario it is just as likely that decoding a bit received by eavesdropper 130 produces an incorrect value as it is that the decode produces the correct value. As used herein, the term “about” can include traditional rounding according to significant figures of numerical values.

Some embodiments of secure physical layer 160 achieve security through the one-half value for p_{EVE} by transforming user data to exploit characteristics that are specific to main channel 110. For example, secure physical layer 160 may exploit one set of characteristics for a wired (also known as wireline) channel and another set for a wireless channel. As another example, secure physical layer 160 may exploit one set of characteristics for a near-field wireless channel, another set for a short-range wireless channel such as WiFi, and yet another set for a long-range wireless channel such as WiMAX. Secure physical layer 160R in secure receiver 120R recovers the originally transmitted user data from the received transformed data by performing the inverse or complement of the particular transform used by secure transmitter 120T.

Some embodiments of secure physical layer 160 achieve security by exploiting the proximity of secure transmitter 120T and secure receiver 120R as compared to eavesdropper 130. When the distance from secure transmitter 120T to secure receiver 120R is much smaller than the distance from secure transmitter 120T to eavesdropper 130, the signal-to-noise ratio on main channel 110 (SNR_{MAIN}) is better than the signal-to-noise ratio on eavesdropper channel 140 (SNR_{EVE}), as can be shown using basic communications theory. Some embodiments of secure transmitter 120T utilize secure error-correction codes, which exploit this difference between SNR_{MAIN} and SNR_{EVE} to insure that information on main channel 110 remains secret from eavesdropper 130 while also providing high reliability on main channel 110.

The use of secure error-correction codes (SECCs) by secure transmitter 120T provides a perfect secrecy zone within a given distance Z from secure transmitter 120T. In some embodiments, the perfect secrecy zone is a circle, so that Z is the radius of that circle. Outside the perfect secrecy zone, the signal-to-noise ratio on eavesdropper channel 140 (SNR_{EVE}) results in a bit error rate on eavesdropper channel 140 (BER_{EVE}) that is high enough to guarantee that a specific percentage of the bits obtained from transmissions by secure transmitter 120T are unreliable. The SECC utilized by secure transmitter 120T guarantees that this unreliable information renders eavesdropper 130 unable to reliably decode messages sent on main channel 110. The SECC is suitably designed to ensure that the bit error rate experienced by the eavesdropper is higher than the bit error rate produced by a conventional error correcting code.

Secure physical layer 160 can be incorporated into a variety of applications and utilized in a variety of environments. One such application is wireless communication by medical devices. Medical data is often considered sensitive and private by the patient, yet wireless communication is vulnerable to interception. Using secure physical layer 160, a medical device can communicate, over a wireless communication channel, patient-related data to other devices and systems located within the secrecy zone. Secure physical layer 160 insures that an eavesdropper outside of this zone cannot recover any data from intercepted transmissions, including patient-related medical data.

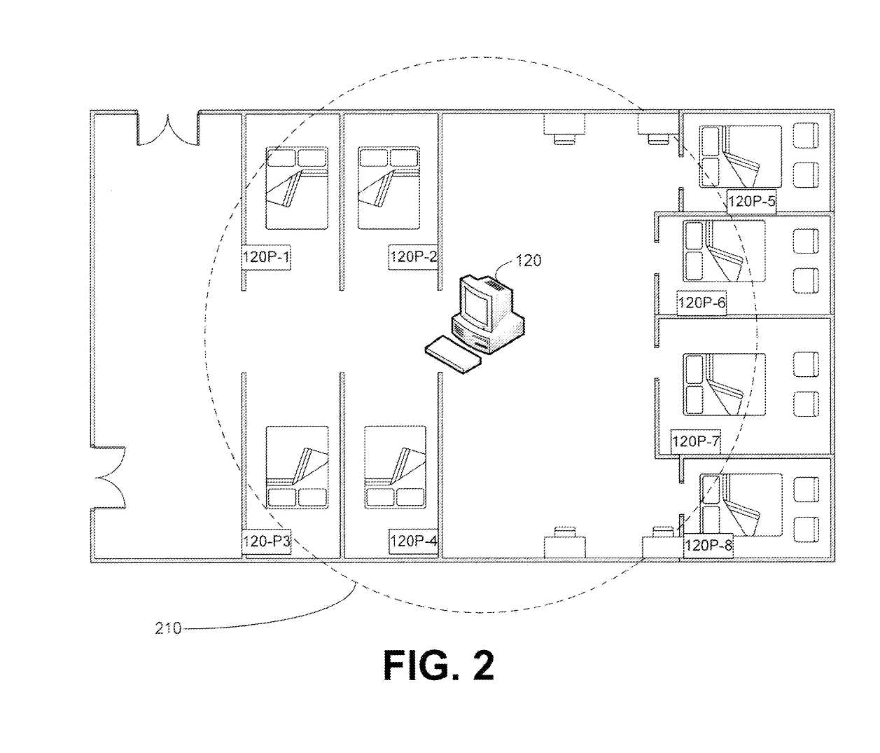

FIG. 2 is a system diagram of a hospital environment that includes medical devices with a secure physical layer 160. A wireless secure communication device 120 communicates with wireless secure communication device 120. The wireless devices 120 and 120P may use various wireless communication technologies, including but not limited to WiFi (IEEE 802.11), Bluetooth, Radio Frequency Identifier (RFID), and Near Field Communications (NFC), to communicate medical data about patient 410. Wireless secure medical device 120P provides a secrecy zone 210, within which wireless secure communication device 120 can securely communicate with various wireless secure medical devices 120P also having a secure physical layer 160. Secure physical layer 160 uses the fact that channel quality degrades as distance from the transmitter increases, and exploits this characteristic to create secrecy zone 210.

In the example environment shown in FIG. 2, wireless secure medical devices 120P-1, 2, 4, 6, and 7 reside within secrecy zone 210 provided by wireless secure communication device 120. Therefore, wireless secure communication device 120 can securely transmit and/or receive medical data associated with patients that are holding, wearing, implanted with, or otherwise physically connected to these medical devices. In contrast, wireless secure medical devices 120P-3, 5, and 8 do not reside within secrecy zone 210, and thus medical data transmitted by those devices is not protected from interception by an eavesdropper.

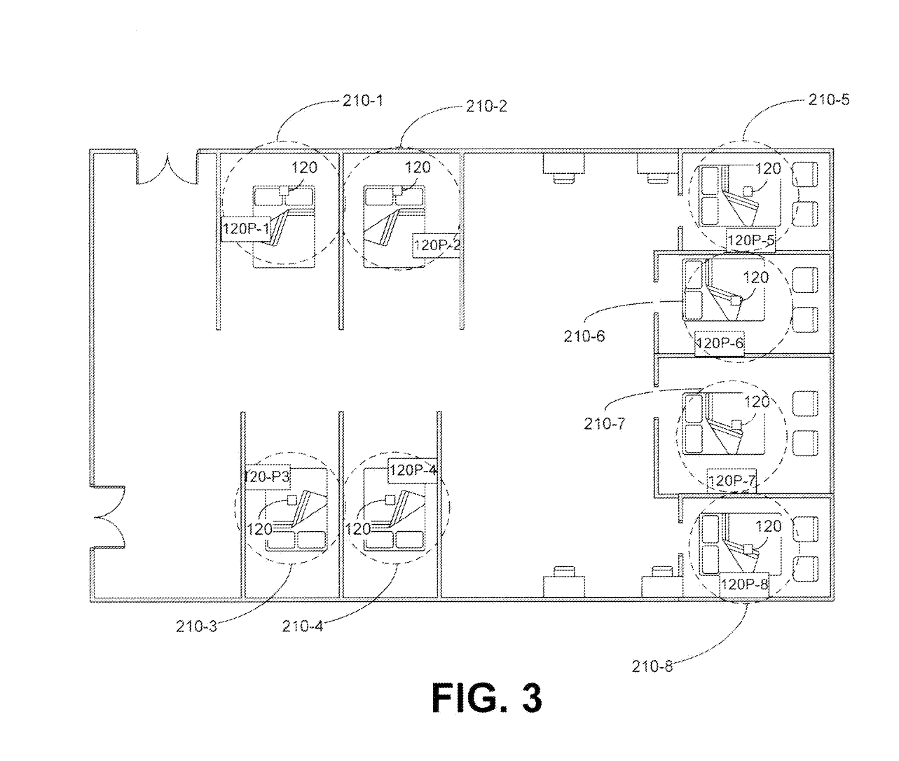

FIG. 3 is a system diagram of another hospital environment that includes medical devices with a secure physical layer 160. In the environment shown in FIG. 3, a site (e.g., an Emergency Room of a hospital) includes multiple secure communication devices 120, each of which provides a corresponding secrecy zone 210. In this example, the size of individual secrecy zones 210 is reduced as compared to the embodiment described in connection with FIG. 2. In this example, each of wireless secure medical devices 120P-1 . . . 8 is paired with a single wireless secure medical device 120P, thus creating multiple local secrecy zones 210-1 . . . 8.

Wireless secure medical devices 120P can take many forms. In some embodiments, medical device 120P is a passive device that senses condition(s) in the patient's body, generates data representative of the condition(s), and transmits this medical data to wireless secure communication device 120. Examples include sensors for pulse, blood pressure, and/or respiration rate, as well as electrocardiogram (EKG) and electroencephalogram (EEG) sensors. In other embodiments, wireless secure medical device 120P is an imaging or other diagnostic device, and wireless secure medical device 120P transmits images or other diagnostic data to wireless secure communication device 120. In still other embodiments, wireless secure medical device 120P is a treatment device that delivers an electric current to the patient (e.g., pacemaker or defibrillator), or that delivers a drug or fluid to the patient (e.g., drug delivery pump, fluid metering device), or on any other device that administers a therapeutic treatment to the patient. In such active devices, data received by wireless secure medical device 120P controls operation of the device so as to administer the treatment to the patient.

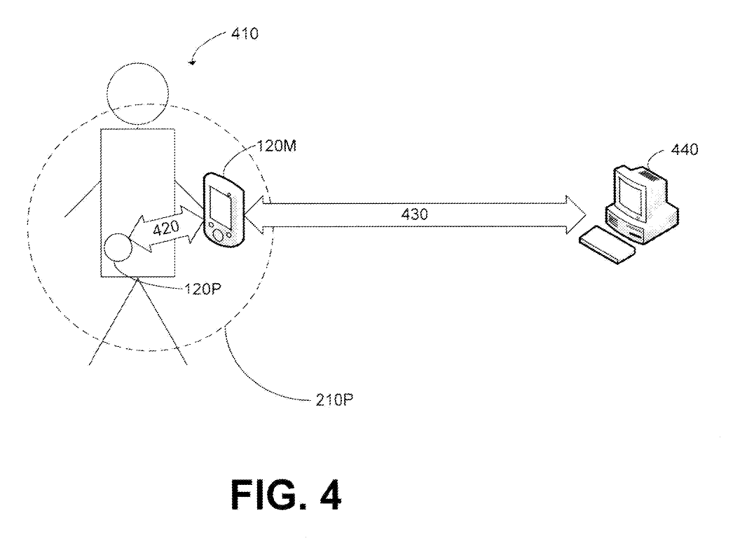

FIG. 4 is a system diagram of another environment that includes a medical device having a secure physical layer 160. In this example, wireless secure communication device 120 takes the form of a wireless secure mobile device 120M. When carried by a patient 410, wireless secure mobile device 120M provides a secrecy zone 210P around the patient's body. Within this secrecy zone 210P, communications between wireless secure mobile device 120M and wireless secure medical device 120P over communication channel 420 are private (free from eavesdropping) and error-free. The wireless devices 120M and 120P may use various wireless communication technologies, including (but not limited to) WiFi (IEEE 802.11), Bluetooth, RFID, and NFC, to communicate medical data about patient 410.

The type of data transmitted to or from wireless secure medical device 120P, and the frequency with which the data is communicated, may vary according to the type of device. For example, if wireless secure medical device 120P is a monitoring device, then wireless secure medical device 120P may periodically report data describing the patient's condition to wireless secure mobile device 120M. As another example, if wireless secure medical device 120P is an active device, then wireless secure medical device 120P may receive instructions for administering a treatment to patient 410. Some embodiments of an active wireless secure medical device 120P receive instructions only once, while others periodically receive instructions from wireless secure mobile device 120M.

After patient condition data is generated by wireless secure medical device 120P and securely received by wireless secure mobile device 120M, wireless secure mobile device 120M forwards patient condition data outside of the local secrecy zone 210P using a second communication channel 430. In the example environment shown in FIG. 4, wireless secure mobile device 120M transmits the patient condition data to a device at a physician's office 440. In another environment, the patient condition data is forwarded to a hospital; however, it should be noted that the remote location is not limited to these particular examples. Since the forwarding location is remote, second communication channel 430 does not typically have physical layer security. However, secondary communication channel 430 may use another form of security, or use security at a different layer. For example, technologies such as Virtual Private Network (VPN), Transport Layer Security (TLS), and other types of encryption may be used when forwarding the patient condition data outside the local secrecy zone 210P.

As noted above, secure physical layer 160 prevents eavesdropping. In some environments, it may be desirable to incorporate additional security measures to militate against active attackers (as compared to eavesdroppers). For example, when wireless secure medical device 120P is an active medical device performing medical procedures for a patient, an attack may attempt to gain control of wireless secure medical device 120P and thus control, interrupt, or otherwise interfere with the treatment. Some embodiments of wireless secure medical device 120P and wireless secure mobile device 120M use a handshake protocol to protect against attackers, in which wireless secure medical device 120P only responds to queries and/or commands received from a wireless secure mobile device 120M within secrecy zone 210P. Some embodiments of wireless secure medical device 120P and wireless secure mobile device 120M utilize secret keys to protect against attacks. In some keyed embodiments, secure physical layer 160 is itself configured by a secret key. Some keyed embodiments use the secret key to perform encryption. Some keyed embodiments use secret keys for both the physical layer 160 and for encryption.

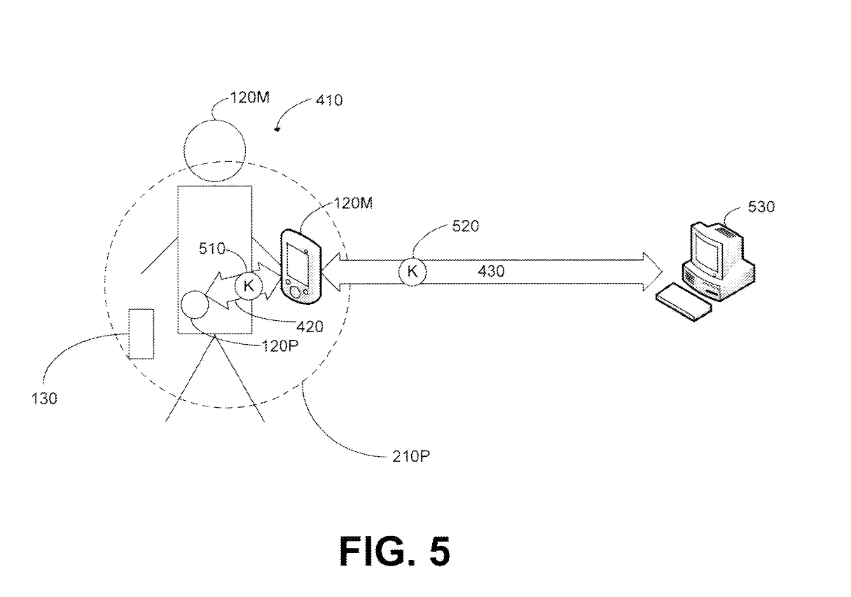

FIG. 5 is a system diagram of an environment in which wireless secure medical device 120P and wireless secure mobile device 120M use secret keys. As with the environment discussed in connection with FIG. 4, wireless secure mobile device 120M provides a secrecy zone 210P around patient 410. As wireless secure medical device 120P is within this zone, wireless secure medical device 120P and wireless secure mobile device 120M are able to exchange patient medical data privately. Such communication also utilizes a secret key 510 shared by wireless secure medical device 120P and wireless secure mobile device 120M. By combining physical layer security and secret keys, communication of patient medical data between wireless secure medical device 120P and wireless secure mobile device 120M is protected from both eavesdroppers 130 and attackers.

The example of FIG. 5 also uses encryption via a secret key 520 when communicating patient medical data over secondary channel 430. Remote site 530 is outside of secrecy zone 210P and thus does not have proximity-based security at the physical layer. However, encryption guards against attacks on wireless secure mobile device 120M or remote site 530.

The use of a key-configured secure physical layer 160, introduced above, will now be discussed in more detail. A key-configured secure physical layer 160 provides error-free communication for secure wireless devices 120M or 120P that reside within secrecy zone 210 and that share a secret key. Receiving devices 120M or 120P that reside within secrecy zone 210 but do not know the secret key can obtain the transmitted data, but because the transmission is not error-free, must compensate for the lack of knowledge to recover the information carried in the transmitted data. In contrast, receiving devices 120M or 120P without knowledge of the secret key, and that are also outside secrecy zone 210, are unable to obtain the transmitted data at all. Such receivers 120M or 120P are thus unable to even attempt a recovery of the information carried within the transmission. Turning now to transmitters, transmitting devices 120M or 120P that do not know the secret key can be detected, since such devices will not be able to properly encrypt transmitted data as expected by the receiver. Finally, use of a handshake protocol prevents transmitting devices 120M or 120P that reside outside of secrecy zone 210 from any communication with wireless secure communication device 120, since the receiver will ignore queries or commands that originate from outside of secrecy zone 210.

As noted above, secure physical layer 160 can be incorporated into a variety of applications and utilized in a variety of environments. One such application for secure physical layer 160 is systems that electronically control access to physical areas such as rooms, buildings, properties, etc. Electronic access systems involve a mobile device, a reader, and an access mechanism. The mobile device communicates with a reader, over a wireless link, to provide credentials to the reader. Once the reader verifies the credentials, the reader controls an access mechanism such as a lock in order to allow access to the secured physical area. Conventional electronic access systems are vulnerable to an eavesdropper intercepting the credential as it is transmitted from the mobile device and the reader. Systems disclosed herein use secure physical layer 160 to render any data obtained by the eavesdropper unusable due to its high error rate.

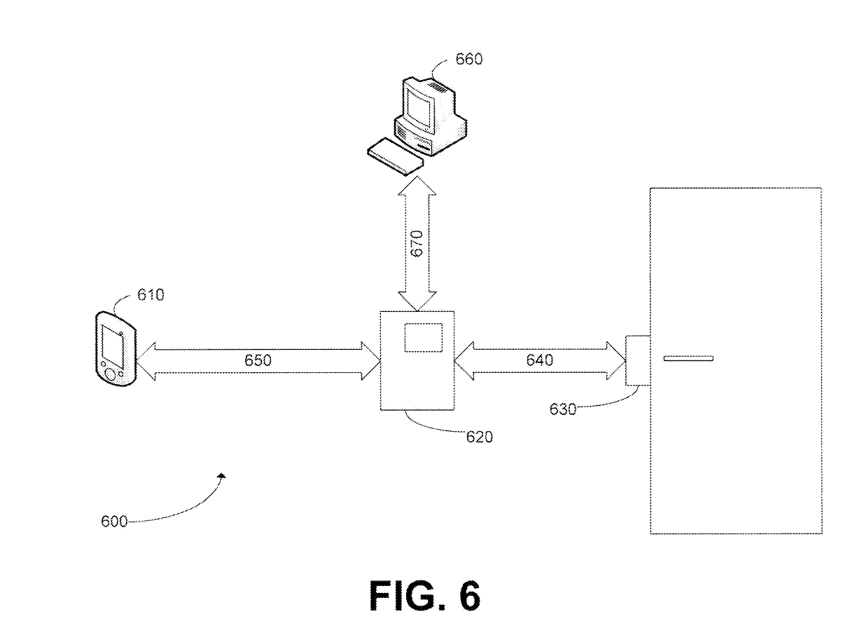

FIG. 6 is a system diagram of an access system utilizing secure physical layer 160. System 600 includes a wireless secure mobile device 610, a secure credential reader 620, and an access mechanism 630. Access mechanism 630 controls access to a restricted area (e.g., door, residence, garage door, hotel room, parking lot, dormitory, resort, commercial building, business suite, automobile, post office box, safe deposit box, public facility, entertainment or sporting facility, transportation facility).

Secure credential reader 620 and wireless secure mobile device 610 each include a secure physical layer 160. As noted above, secure physical layer 160 prevents an eavesdropper from recovering transmitted data by insuring that the eavesdropper experiences a high error rate. In some embodiments, secure physical layer 160 guarantees that an eavesdropper experiences a bit error rate (BER) equal or close to 0.5.

Notably, the secure physical layer 160 in wireless secure mobile device 610 is paired with the secure physical layer 160 in secure credential reader 620. A secret key shared by secure credential reader 620 and wireless secure mobile device 610 permit secure credential reader 620 and wireless secure mobile device 610 to communicate with each other, but does not allow communication with any device that does not also possess the secret key.

Secure credential reader 620 controls access mechanism 630 through signals delivered over link 640, instructing access mechanism 630 to allow access to (unlock) the restricted area or to disallow access to (lock) the restricted area. Secure credential reader 620 decides whether to unlock the restricted area based on a digital credential provided by wireless secure mobile device 610 over a wireless secure channel 650. The digital credential can include a self-destruct feature, whereby the credential is no longer valid after a predefined amount of time.

When a user desires access to restricted area, the user places a wireless secure mobile device 610 in the vicinity of secure credential reader 620. Wireless secure mobile device 610 can take a variety of forms, including (but not limited to) a smart card, a Radio Frequency Identification (RFID) tag, an NFC tag, and a mobile phone. Wireless secure mobile device 610 transmits a digital credential to secure credential reader 620. Secure credential reader 620 determines whether the mobile-provided digital credential matches a list of credentials that are allowed access to the restricted area. If the mobile-provided credential is verified, then secure credential reader 620 signals access mechanism 630 to unlock; if, however, the mobile-provided credential fails verification, then secure credential reader 620 does not signal access mechanism 630 to unlock.

In the embodiment shown in FIG. 6, secure credential reader 620 sends a verification request to a verifier 660 over a channel 670, and verifier 660 responds with an indication as to whether the credential was verified or not. In other embodiments, secure credential reader 620 itself performs the comparison of the mobile-provided digital credential and the list of authorized credentials.

Various technologies can be used for link 640, channel 650, and channel 670. Wireless technologies which may be used to implement channel 650 include Bluetooth, Near Field Communication (NFC), and Radio Frequency Identification (RFID). Channel 670 may use wire-line (wired) technologies such as Ethernet, Universal Serial Bus (USB), or may use wireless technologies such as WiFi, WiMAX, and Bluetooth. Link 640 may be wired, e.g., Inter-Integrated Circuit (I²C) or Controller Area Network (CAN) bus, or may use a wireless technology. In addition, link 640 and channel 670 may optionally utilize a secure physical layer 160, to prevent eavesdropping on these links also.

As described above, secure physical layer 160 in secure credential reader 620 is paired with secure physical layer 160 in wireless secure mobile device 610 so that the layers share the same secret key. Various mechanisms can be used to configure the secure physical layers 160 in the corresponding devices 610 and 620 with a secret key. For example, the user of wireless secure mobile device 610 may input a personal identification number (PIN), password, or password. The user-provided information is then used to generate a secret key, and the secret key is provided to both wireless secure mobile device 610 and secure credential reader 620. As another example, a remote host can generate a secret key and transmit the key to wireless secure mobile device 610. The configuration of a wireless secure mobile device 610 with a secret key can be static (e.g., performed during a manufacturing or provisioning procedure) or dynamic (e.g., on demand as requested by a user). The key used for configuration can include a self-destruct feature, whereby the key is no longer valid after a predefined amount of time.

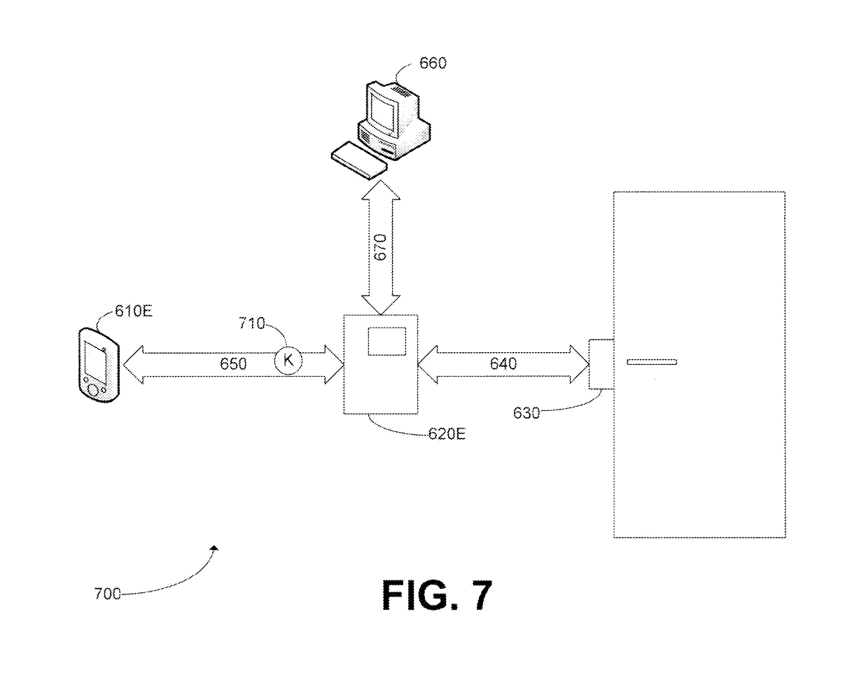

As noted above, secure physical layer 160 within secure credential reader 620 and wireless secure mobile device 610 prevents eavesdropping. In some environments, it may be desirable to incorporate additional security measures to militate against active attackers (as compared to eavesdroppers). FIG. 7 is a system diagram of an access system with secure physical layer 160 and which uses a cryptographic algorithm to encrypt the credential. Access system 700 includes a wireless secure mobile device 610E, a secure credential reader 620E, an access mechanism 630, and a verifier 660. Wireless secure mobile device 610P communicates with secure credential reader 620 over wireless channel 650, secure credential reader 620 communicates with access mechanism 630 over link 640, and secure credential reader 620 communicates with verifier 660 over channel 670. Wireless secure mobile device 610P encrypts the user's credential with a secret key 710 before transmitting the credential to secure credential reader 620P over wireless channel 650. In some embodiments (not shown), encryption is also used on channel 670 between secure credential reader 620 communicates with verifier 660. In some embodiments, encryption is also used on link 640 between secure credential reader 620 and access mechanism 630. The techniques described herein can be used to implement a standalone solution for electronic access systems that do not already have encryption, or as an add-on to existing systems that already employ cryptography, thus further enhancing the overall security of the access system.

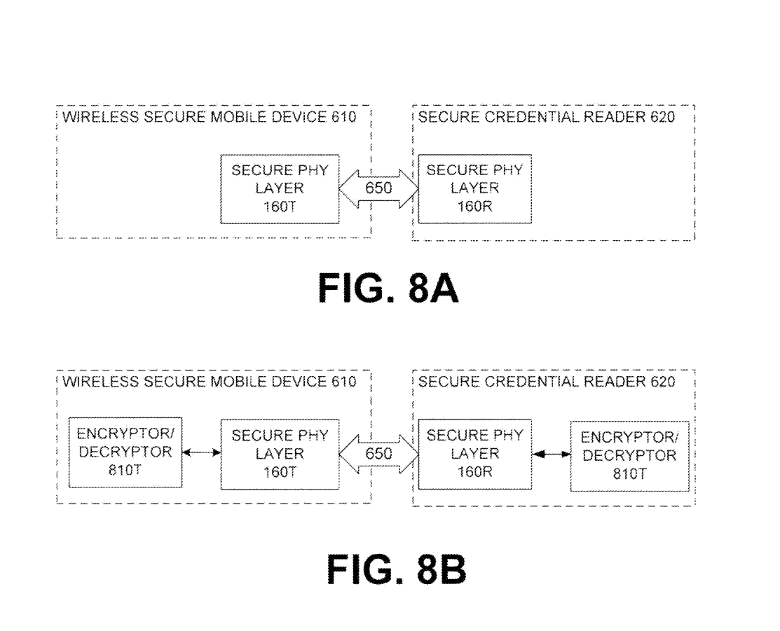

FIGS. 8A and B are system diagrams of access systems utilizing secure physical layer 160. FIG. 8A illustrates an embodiment without encryption. In this standalone secure physical layer embodiment, the secure physical layer 160M of wireless secure mobile device 610 communicates with the secure physical layer 160R of secure credential reader 620 over wireless channel 650. Although credential data transmitted over this channel 650 is private, it is not encrypted. However, to enhance privacy, the secure physical layer 160 can be configured with keys as described before. FIG. 8B illustrates an embodiment which also includes encryption of credentials. In this combined secure physical layer and cryptography embodiment, an encryption/decryption module 810 in wireless secure mobile device 610M encrypts the mobile device credential, and this encrypted credential is transmitted by the secure physical layer 160M over wireless channel 650. One received and processed by secure physical layer 160R, the credential is then decrypted by encryption/decryption module 810. Credential data transmitted over this channel 650 is thus both private (protected from eavesdropping) and encrypted.

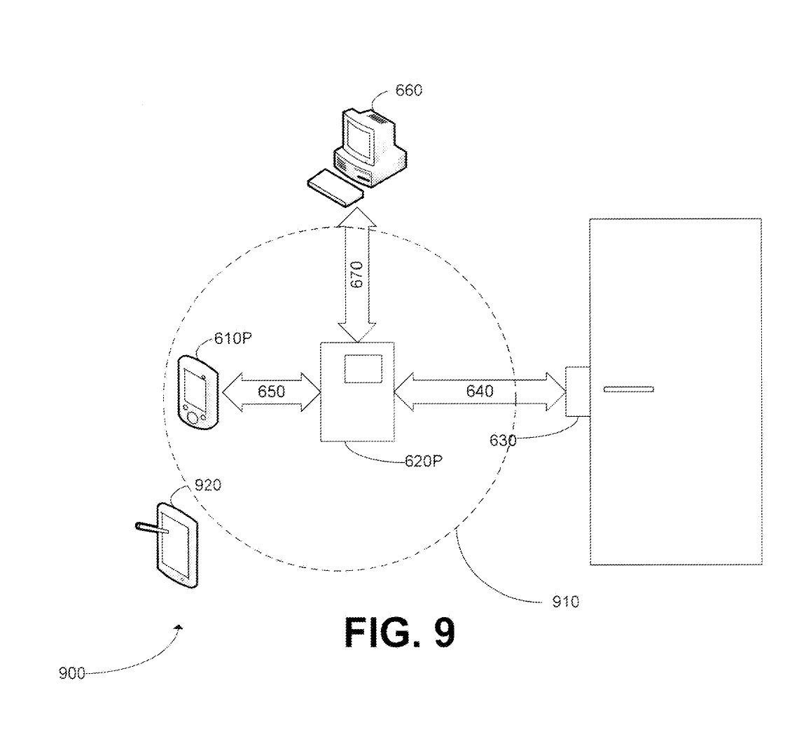

FIG. 9 is a system diagram of an access system utilizing secure physical layer 160 that provides proximity-based security at the physical layer. Access system 900 includes a wireless secure mobile device 610P, a secure credential reader 620P, an access mechanism 630, and a verifier 660. Wireless secure mobile device 610P and secure credential reader 620P each include a secure physical layer 160 (not shown) that provides a secrecy zone 910 around the transmitter. An eavesdropper 920 outside of secrecy zone 910 is unable to reliably recover information from data transmitted within secrecy zone 910. Thus, transmission of the credential from wireless secure mobile device 610P to secure credential reader 620P is guaranteed to be private.

As noted above, secure physical layer 160 can be incorporated into a variety of applications and utilized in a variety of environments. One such application for secure physical layer 160 is location-based marketing systems. Location-based marketing systems typically allow a customer to share identifying credentials with a merchant on entering a building, and in return provide the customer with better offers and coupons through loyalty status or rewards programs. Unlike conventional systems, the location-based marketing systems disclosed herein use secure physical layer 160 to render any data obtained by the eavesdropper unusable due to its high error rate.

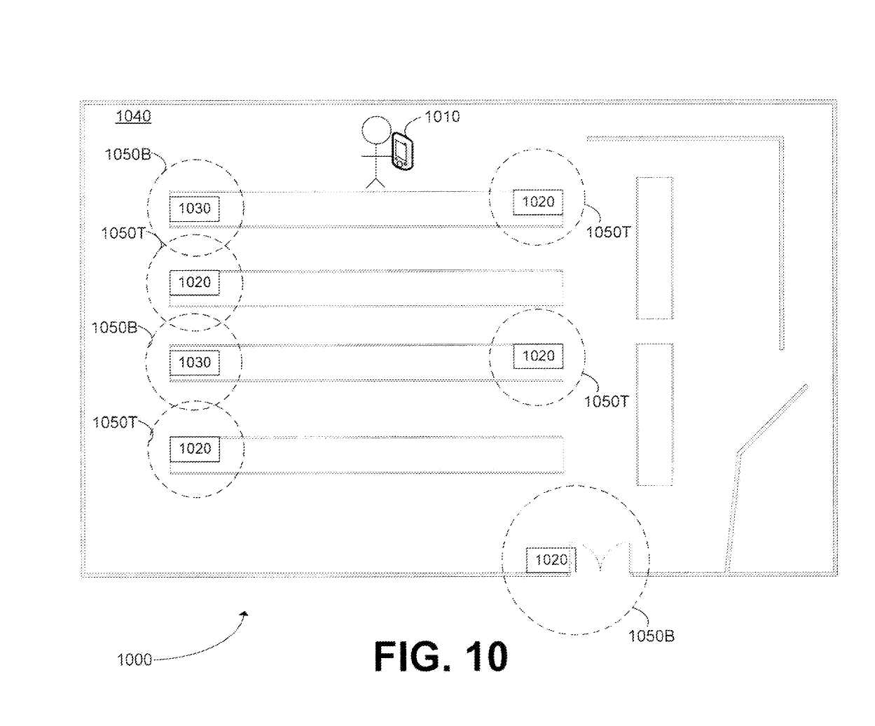

FIG. 10 is a system diagram of a location-based marketing system utilizing secure physical layer 160. System 1000 includes one or more wireless secure mobile devices 1010, one or more wireless tags 1020, one or more wireless beacons 1030, and a location-based marketing server (not shown). Wireless tags 1020 are located throughout a site 1040, for example, a store or other retail establishment. Some embodiments of wireless tag 1020 may be implemented with smart tag (also known as smart label) technology. In some embodiments, wireless tags 1020 are attached to or otherwise near products, goods, or merchandise. In some embodiments, wireless tags 1020 are attached to, or included in, a poster or print advertisement. Wireless secure mobile devices 1010, wireless beacons 1030, and wireless tags 1020 communicate among themselves using a wireless technology such as Bluetooth, RFID, or NFC. Wireless secure mobile devices 1010, wireless beacons 1030, and wireless tags 1020 communicate with location-based marketing server using a wired or a wireless technology. A non-limiting list of wireless technologies used by location-based marketing server includes Bluetooth, WiFi, and WiMAX.

As a user with a wireless secure mobile device 1010 travels through site 1040 and passes in the vicinity of a particular wireless tag 1020, information is communicated between the wireless tag 1020 and wireless secure mobile device 1010. In some embodiments, the wireless tag 1020 provides information to wireless secure mobile device 1010 about the product associated with the wireless tag 1020. Wireless beacons 1030 also reside at various locations within site 1040.

When a user with a wireless secure mobile device 1010 passes in proximity to a particular wireless beacon 1030, the wireless beacon 1030 determines the identity of wireless secure mobile device 1010, for example, through a digital credential. Together, wireless beacons 1030 allow the location of a user to be tracked as the user's wireless secure mobile device 1010 moves through site 1040. Wireless beacons 1030 may report the movement of wireless secure mobile device 1010 to location-based marketing server. By identifying a user through his digital credentials and then combining loyalty program information, user preference, and/or user behavior information with current user location and product location information, location-based marketing server can provide the user with specifically targeted offers and coupons. Offers and coupons targeted in this manner are likely to be perceived as relevant by the user.

Wireless secure mobile devices 1010, wireless beacons 1030, and wireless tags 1020 communicate among each other using wireless technology, and wireless technologies are generally vulnerable to eavesdropping. To address this vulnerability, each of wireless secure mobile devices 1010, wireless beacons 1030, and wireless tags 1020 includes a secure physical layer 160 which provides privacy. These secure physical layers 160 use the fact that channel quality degrades as distance from the transmitter increases, and exploit this characteristic to provide a secrecy zone around their respective transmitting devices. More specifically, each wireless tag 1020 provides a tag secrecy zone 1050T. In some the embodiments shown in FIG. 10, each wireless beacon 1030 also provides a beacon secrecy zone 1050B. Data transmitted within tag secrecy zone 1050T, such as product information and user credentials is thus protected from eavesdroppers outside of tag secrecy zone 1050T. Similarly, data transmitted within beacon secrecy zone 1050B, such as user credentials, is protected from eavesdroppers outside of beacon secrecy zone 1050B.

Without the privacy provided by secure physical layers 160 within system 1000, customer-specific data could be compromised by an eavesdropper, attackers could impersonate other customers and obtain better offers, and attackers could falsify data provided to the merchant by shopping under an improper marketing credential. Because system 1000 provides privacy for transmitters within various secrecy zones 1050, customers can feel confident in sharing an identifying credential with a merchant. Merchants can be confident that personalized offers and coupons targeted at a particular user are obtained only by that user and not by other shoppers.

As noted above, secure physical layer 160 within wireless secure mobile devices 1010, wireless beacons 1030, and wireless tags 1020 prevents eavesdropping. In some environments, it may be desirable to incorporate additional security measures to militate against active attackers (as compared to eavesdroppers). Some embodiments of wireless secure mobile device 1010 use a handshake protocol to protect against attackers, in which wireless secure mobile device 1010 only responds to queries and/or commands received from a wireless beacon 1030 or wireless tag 1020 that resides within a corresponding secrecy zone 1050. Some embodiments of utilize secret keys to protect against attacks. In some keyed embodiments, secure physical layer 160 is itself configured by a secret key. Some keyed embodiments use the secret key to perform encryption.

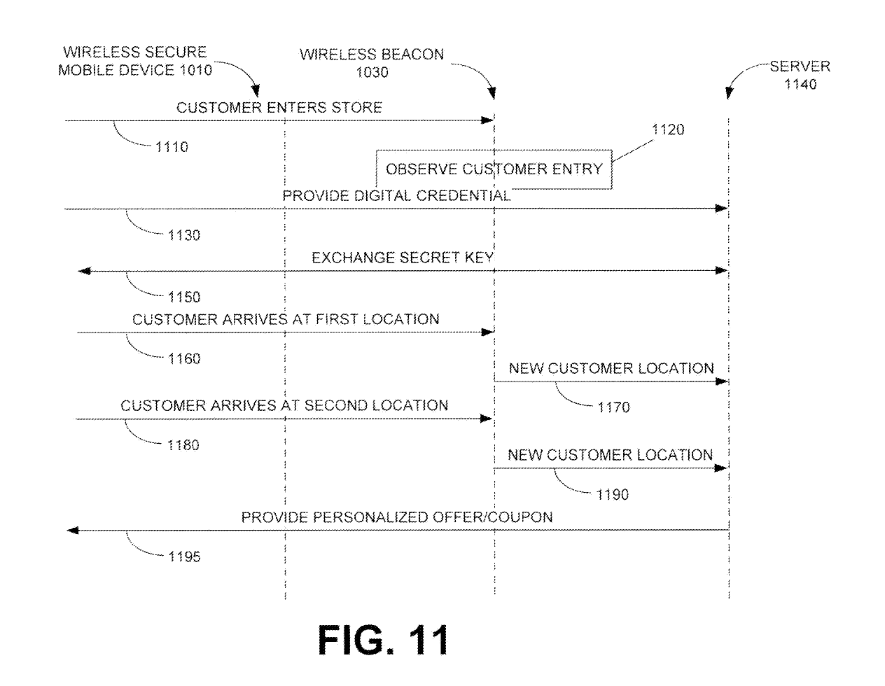

FIG. 11 is a messaging diagram illustrating operation of various components in one embodiment of system 1000. At time point 1110, a customer uses wireless secure mobile device 1010 to check in with a merchant when entering a store. The customer's entry may be observed (block 1120) by a wireless beacon 1030 placed at the store entrance. During this check in procedure, wireless secure mobile device 1010 shares (via message 1130) a digital credential with location-based marketing server 1140. This digital credential identifies the user and links the user to a loyalty or rewards program associated with the merchant. This communication utilizes secure physical layer 160 to ensure privacy. In this manner, eavesdroppers that are outside of the secrecy zone 1050 (FIG. 10) provided by the checkpoint cannot obtain any information about the user's credential. During the check in procedure, a secret key is shared (via message 1150) between wireless secure mobile device 1010 and location-based marketing server 1140. In some embodiments, the check in procedure uses a handshake protocol that is protected by secure physical layer 160.

At time point 1160, the customer arrives at a first location within site 1040 and this position is observed by a wireless beacon 1030. The observing wireless beacon 1030 notifies location-based marketing server (via message 1170) of the customer's location. At time point 1180, the customer moves to a second location within site 1040. This new position is observed by a different wireless beacon 1030. The second wireless beacon 1030 notifies location-based marketing server (via message 1190) of the customer's new location. A location-based marketing server then transmits (via message 1195) a personalized offer or coupon to the customer via a wireless channel, for example, WiFi.

In some embodiments, the transmission of the offer or coupon uses a time sharing protocol. This allows many offers and coupons to be transmitted substantially simultaneously, while at the same time each offer/coupon is secured at the physical layer by the secret key agreed upon during the check in procedure. To allow multiple customers to obtain offers/coupons in tandem, the secrecy zone 1050 that location-based marketing server provides to protect offer/coupon transmission may be much larger than the secrecy zone 1050 provided by a wireless beacon 1030, or than the secrecy zone 1050 provided by wireless tag 1020. However, even with a larger server secrecy zone 1050, the shared secret key prevents customers from viewing or obtaining offers meant for others.

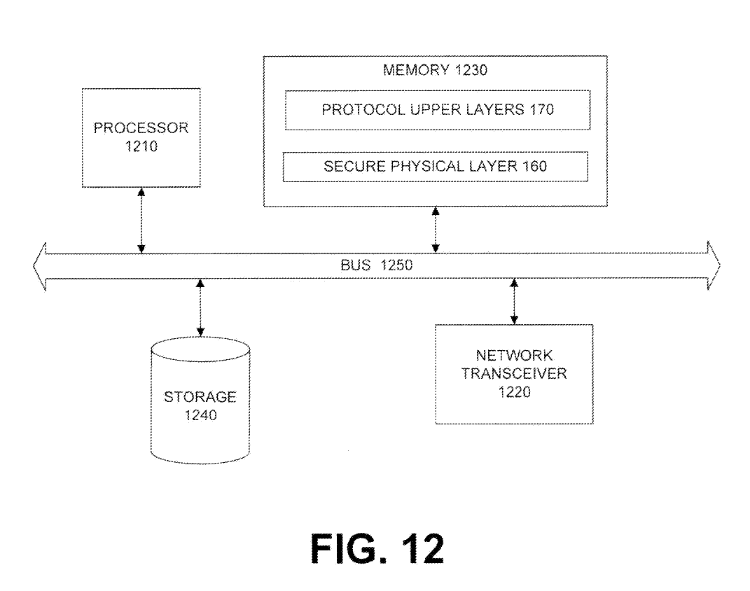

FIG. 12 is a hardware block diagram of an embodiment of secure communication device 120 in which secure physical layer 160 is implemented in software or firmware. Secure communication device 120 contains a number of components that are well known in the art of data communications, including a processor 1210, a network transceiver 1220, memory 1230, and non-volatile storage 1240. These components are coupled via a bus 1250. In this software embodiment, secure physical layer 160 is implemented as instructions stored in a memory and executed by processor 1210, which may be implemented as a microprocessor, digital signal processor, network processor, microcontroller, etc. In this embodiment, instructions for protocol upper layers 170 are also stored as instructions in memory 1230.

Network transceiver 1220 may support one or more of a variety of different networks using various technologies, medias, speeds, etc. A non-limiting list of examples of wireless technologies includes: radio frequency identification (RFID) networks (e.g., ISO 14443, ISO 18000-6); wireless local area networks (e.g. IEEE 802.11, commonly known as WiFi); wireless wide area networks (e.g., IEEE 802.16, commonly known as WiMAX); wireless personal area networks (e.g., Bluetooth™, IEEE 802.15.4) and wireless telephone networks (e.g., CDMA, GSM, GPRS, EDGE).

Examples of non-volatile storage include, for example, a hard disk, flash RAM, flash ROM, EPROM, etc. Memory 1230 contains security transformer instructions 1260 and/or inverse security transformer instructions 1270, which programs or enables processor 1210 to implement the functions of secure physical layer 160. Omitted from FIG. 12 are a number of conventional components, known to those skilled in the art, that are not necessary to explain the operation of secure communication device 120.

Some embodiments of secure physical layer 160 are stored on a computer-readable medium, which in the context of this disclosure refers to any structure which can contain, store, or embody instructions executable by a processor. The computer readable medium can be, for example but not limited to, based on electronic, magnetic, optical, electromagnetic, infrared, or semiconductor technology. Specific examples of a computer-readable medium using electronic technology would include (but are not limited to) the following: a random access memory (RAM); a read-only memory (ROM); and an erasable programmable read-only memory (EPROM or Flash memory). A specific example using magnetic technology includes (but is not limited to) a disk drive; and a portable computer diskette. Specific examples using optical technology include (but are not limited to) a compact disk read-only memory (CD-ROM) or a digital video disk read-only memory (DVD-ROM).

Other embodiments of secure physical layer 160 (not illustrated) are implemented in hardware logic, as secure physical layer logic. Technologies used to implement security transformer logic and inverse security transformer logic in specialized hardware may include, but are not limited to, a programmable logic device (PLD), a programmable gate array (PGA), field programmable gate array (FPGA), an application-specific integrated circuit (ASIC), a system on chip (SoC), and a system on packet (SoP). In yet another embodiment of secure communication device 120 (not illustrated), secure physical layer 160 is implemented by a combination of software (i.e., instructions executed on a processor) and hardware logic.

1. A system comprising:
a medical sensor device operable to generate data representative of a condition of a patient;
a wireless communication module operable to transmit, on a wireless communication channel, the generated data representative of the condition of the patient; and
a physical layer security module residing at a physical layer of the wireless communication module and operable to provide a secrecy zone around the physical layer security module by transforming the generated data such that transmission of the generated data is secured from interception by an eavesdropper on the wireless communication channel.

2. The system of claim 1, wherein the physical layer security module is further operable to transform the data representative of the condition of the patient in accordance with one or more security characteristics, the one or more security characteristics operating to provide a bit error rate of about one-half when the data as intercepted by an eavesdropper on the wireless communication channel is decoded, thereby providing the secrecy zone.

3. The system of claim 1, wherein the physical layer security module is further operable to transform the user data by encoding the user data with a secure error correction code.

4. The system of claim 1, wherein the wireless communication module is further operable to perform error correction coding on the transformed data.

5. The system of claim 1, wherein the sensor is located in proximity to the patient.

6. The system of claim 1, wherein the sensor is affixed to the patient.

7. The system of claim 1, wherein the sensor is implanted in the patient.

8. A system comprising:
a wireless communication module operable to receive, on a wireless communication channel, control data associated with a therapeutic treatment;
a physical layer security module residing at a physical layer of the wireless communication module and operable to provide a secrecy zone around the physical layer security module; and
a medical device operable to administer the therapeutic treatment to a patient in accordance with the received control data associated with the therapeutic treatment.

9. The system of claim 8, wherein the control data includes one or more parameters describing the therapeutic treatment.

10. The system of claim 8, wherein the control data includes an identifier of the therapeutic treatment.

11. The system of claim 8, wherein the medical device is affixed to the patient.

12. The system of claim 8, wherein the medical device is implanted in the patient.

13. The system of claim 8, wherein the medical device is operable to provide an electric current to the patient in accordance with the received control data.

14. The system of claim 8, wherein the medical device is operable to administer a drug to the patient in accordance with the received control data.

15. A system comprising:
a wireless mobile communication device having a credential stored thereon and operable to communicate the credential over a wireless communication channel;
an access mechanism operable to control access to a protected area; and
a reader operable to receive the credential from the wireless mobile communication device over the wireless communication channel, to request a verification of the received credential, and to instruct the access prevention mechanism to allow access to the protected area in response to receiving the verification,
wherein the wireless mobile communication device comprises:
a physical layer security module residing at a physical layer of the wireless mobile communication device and operable to transform the credential in accordance with one or more security characteristics that provide a bit error rate of about one-half when the credential as intercepted by an eavesdropper on the wireless communication channel is decoded; and
a wireless communication module operable to transmit the transformed credential to the reader over the wireless communication channel.

16. The system of claim 15, wherein the wireless mobile communication device is further operable to receive a first key and to configure the physical layer security module in accordance with the first key.

17. The system of claim 16, wherein the reader is further configured to receive a second key that is identical to the first key.

18. The system of claim 15, wherein the wireless mobile communication device is further configured to encrypt the credential before providing the credential to the physical layer security module.

19. The system of claim 15, wherein the wireless mobile communication device corresponds to a phone, a smart card, or a tag.

20. The system of claim 15, wherein the wireless communication channel corresponds to a Bluetooth channel, a near field communication (NFC) channel, or a radio frequency identification (RFID) channel.

21. A system comprising:
a wireless mobile communication device having a credential stored thereon and operable to communicate the credential over a wireless communication channel; and
a location-based marketing server operable to receive the credential from the wireless mobile communication device and to select a personalized offer or a personalized coupon based at least in part upon the received credential; and
wherein the wireless mobile communication device comprises:
a physical layer security module residing at a physical layer of the wireless mobile communication device and operable to transform the credential in accordance with one or more security characteristics that provide a bit error rate of about one-half when the credential as intercepted by an eavesdropper on the wireless communication channel is decoded; and
a wireless communication module operable to transmit the transformed credential to the location-based marketing server over the wireless communication channel.

22. The system of claim 21, further comprising a wireless beacon operable to detect a presence of the wireless mobile communication device, wherein the wireless mobile communication device is further operable to use the physical layer security module to communicate with the wireless beacon.

23. The system of claim 21, further comprising a wireless tag, wherein the wireless mobile communication device is further operable to use the physical layer security module to receive product information from the wireless tag.
